(19) United States
(12) Plant Patent Application Publication
(10) Pub. No.: US 2025/0374840 P1
(43) Pub. Date: Dec. 4, 2025

(54) Ilex plant named 'BLHO02'

(50) ;

(71) Applicant: Robert Edward Lee, Independence, LA (US)

(72) Inventor: Robert Edward Lee, Independence, LA (US)

(73) Assignee: Plant Development Services Inc, Loxley, AL (US)

(21) Appl. No.: 18/675,224

(22) Filed: May 28, 2024

(51) Int. Cl. A01H 6/00; A01H 5/12

(52) U.S. Cl. CPC A01H 6/00; A01H 5/12
USPC PLT/247

(57) ABSTRACT

A new and distinct variety of Ilex plant named ‘BLHO02’ is disclosed, characterized by a very compact plant form with dense growth. Plants of ‘BLHO02’ have been observed to have a mature size of 2 feet wide and 2 feet tall. Pruning is not needed to maintain and attractive landscape plant. Foliage and very small and dark green when mature. The new cultivar is an Ilex, suitable for horticultural purposes.

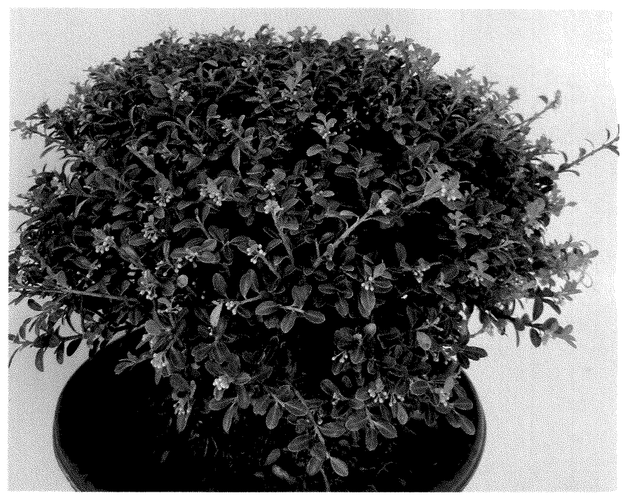

Latin name of the genus and species: Ilex compacta.

Variety denomination: ‘BLHO02’.

### BACKGROUND OF THE INVENTION

The new cultivar, hereinafter referred to as ‘BLHO02’, was discovered as part of a breeding program at a commercial nursery in Independence, LA. The intention of the breeding program was to develop new Ilex varieties with novel plant habits for commercial ornamental purposes. This new discovery is the result of an open-pollination of various unnamed, unpatented proprietary Ilex compacta varieties conducted by the inventor between 2006 and 2008. Plants of various proprietary varieties of Ilex compacta were planted in close proximity in a field to allow open-pollination between these parent varieties. Seeds were bulk-collected between 2006 and 2008. The exact collection year of the new variety cannot be specified, however, it is the inventor's assumption that it was 2006.

‘BLHO02’ was selected as a seedling in the Fall of 2010 at the same commercial nursery in Independence, LA. After identifying the new variety as a potentially interesting selection, the inventor first organized propagation of ‘BLHO02’ by softwood cuttings at the same commercial nursery during the Fall of 2010. The inventor continued controlled testing and propagation, assessing stability of the unique characteristics of this variety. At least four generations have been reproduced and have shown that the unique features of this cultivar are stable and reproduced true to type.

### SUMMARY OF THE INVENTION

The cultivar ‘BLHO02’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘BLHO02’ These characteristics in combination distinguish ‘BLHO02’ as a new and distinct Ilex cultivar:
1. Very compact plant form.
2. Mature height and width 2 feet.
3. Dense growth.
4. Very small foliage.
5. Dark green mature foliage.
6. No pruning required to maintain an attractive plant shape.

### COMPARISON TO PARENT VARIETY

The exact parent varieties cannot be identified.

### COMMERCIAL COMPARISON

Ilex ‘BLHO02’ can be compared to the commercial variety Ilex crenata ‘Compacta’, unpatented. Plants are similar in most horticultural characteristics; however, plants of the new variety differ in the following:
1. Mature height of ‘BLHO02’ is about 2 feet; ‘Compacta’ has a mature height of 4 to 6 feet.
2. Mature width of ‘BLHO02’ is about 2 feet; ‘Compacta’ has a mature width of 4 to 6 feet.
3. Foliage of the new variety is smaller than foliage of this comparator.

Ilex ‘BLHO02’ can be compared to the commercial variety Ilex crenata ‘Soft Touch’, unpatented. Plants are similar in most horticultural characteristics; however, plants of the new variety differ in the following:
1. Mature height of ‘BLHO02’ is about 2 feet; ‘Soft Touch’ has a mature height of 3 feet.
2. Mature width of ‘BLHO02’ is about 2 feet; ‘Soft Touch’ has a mature width of 3 feet.
3. Foliage of the new variety is stiffer/firmer, not as soft as foliage of this comparator.

### BRIEF DESCRIPTION OF THE PHOTOGRAPH

The accompanying photograph illustrates in full color a typical plant of ‘BLHO02’ grown outdoors in Loxley, AL. The plant is approximately 1.5 years old, in a 2-gallon pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘BLHO02’ plants grown outdoors in Loxley, AL. Plants are approximately 1.5 years old, in a 2-gallon nursery container. Measurements and numerical values represent averages of typical plant types.
Botanical classification: Ilex compacta ‘BLHO02’.

### PROPAGATION

Typically by semi-hardwood cuttings in Spring and Summer.
Root description: Semi-woody, moderately dense roots. Colored near RHS Grey-Brown 199A.

### PLANT

Growth habit: Globular evergreen shrub. Low growing.
Height: Approximately 21 cm.
Plant spread: Approximately 28 cm.
Branching characteristics: Well branched, about 4 primary branches from the center of the plant. Each primary branch has 7 to 12 lateral branches. Branches occur at approximately 60° to 90° angles.
Primary branches:
Length of primary branches: Average range 6 to 9 cm, then branching into further laterals.
Diameter of primary branches: Average range 12 mm to 28 mm.
Primary branch strength: Very strong.
Primary branch color: Near RHS Greyed-Green 189A.
Primary branch texture: Rough, slightly scaly.
Lateral branches:
Number of lateral branches: Approximately 7 to 12 per primary branch.
Length of lateral branches: Average range 10 to 25 cm.
Diameter of lateral branches: Average 5 mm 7 mm.
Lateral Branch Strength: Moderately strong.

16 Lateral branch color: Near RHS Grey-Brown 199A with overlay of Greyed-Green 189A.
Lateral branch texture: Glabrous, rough.
Internode length: Average 2 to 3 mm.
Age of plant described: Approximately 1.5 years.

### FOLIAGE

Leaf:
   Type.—Simple.
   Arrangement.—Alternate.
   Average length.—Approximately 1.0 to 1.5 cm.
   Average width.—Approximately 5 to 7 mm.
   Shape of blade.—Oblanceolate.
   Apex.—Obtuse.
   Base.—Attenuate.
   Attachment.—Stalked.
   Margin.—Entire.
   Texture of top surface.—Glabrous.
   Texture of bottom surface.—Glabrous.
   Appearance of top surface.—Glossy.
   Appearance of bottom surface.—Matte.
   Color.—Young foliage upper side: RHS Yellow-Green 144A. Young foliage under side: Near RHS Yellow-Green 144C. Mature foliage upper side: RHS Green 139A. Mature foliage under side: Near RHS Green 137C.
Venation:
   Type.—Pinnate.
   Venation color upper side.—Yellow-Green 145A.
   Venation color under side.—Yellow-Green 145A.
Petiole: No.

### FLOWER

Axillary flowers. Flowering begins around early May in Southern California.
Flower size:
   Diameter.—3 mm.
   Depth.—3 mm.
Peduncle:
   Length.—2 mm.
   Diameter.—1 mm.
   Color.—Near Green 143A.
   Texture.—Glabrous.
Petals:
   Petal arrangement.—Cruciform.
   Length.—0.15 mm.
   Width.—2 mm.
   Shape.—Orbicular.
   Margin.—Entire.
   Apex.—Rounded.
   Base.—Broad cuneate.
   Petal quantity.—4.
   Texture.—Glabrous all surfaces.
   Appearance.—Matte all surfaces.
   Color.—When opening: Upper surface: Near RHS Green-White 157D. Lower surface: Near RHS Green-White 157A. Fully opened: Upper surface: Near RHS White 155C. Lower surface: Near RHS Green-White 157D.
Bud:
   Shape.—Globular.
   Length.—Approximately 1 mm.
   Diameter.—Approximately 1 mm.
   Color.—Near RHS Yellow 2D.
Sepals:
   Quantity.—4.
   Length.—0.5 mm.
   Width.—0.3 mm.
   Shape.—Deltate.
   Color.—Interior Surface: Near RHS Green 143C. Exterior Surface: Near RHS Green 143A.
   Texture.—All surfaces glabrous.
Fragrance: None detected.
Reproductive organs: Minute and indistinguishable.

### OTHER CHARACTERISTICS

Disease and Pest Resistance: Neither resistance nor susceptibility to normal diseases and pets of Ilex observed.
Drought Tolerance: No observed susceptibility for diseases and pests.
Temperature Tolerance: USDA Zones 5 through 9.

What is claimed is:

1. A new and distinct cultivar of Ilex plant named ‘BLHO02’ as herein illustrated and described.
